(19) United States
(12) Patent Application Publication
(10) Pub. No.: US 2025/0345482 A1
(43) Pub. Date: Nov. 13, 2025

(54) 3D PRINTED GALLIUM SCAFFOLD

(71) Applicant: Northeast Ohio Medical University, Rootstown, OH (US)

(72) Inventor: Moses O. Oyewumi, Rootstown, OH (US)

(21) Appl. No.: 19/202,436

(22) Filed: May 8, 2025

Related U.S. Application Data
(60) Provisional application No. 63/643,954, filed on May 8, 2024.

(51) Int. Cl. A61L 27/30; A61L 27/18

(52) U.S. Cl. CPC A61L 27/306; A61L 27/18; A61L 2430/02

(57) ABSTRACT

A biocompatible 3D-printed scaffold is described. The scaffold includes a biocompatible polymer shaped to form a scaffold using 3D printing and a gallium compound. Methods of making 3D-printed scaffolds, and methods of using 3D-printed scaffolds to inhibit bone resorption are also described.

### CROSS-REFERENCE TO RELATED APPLICATIONS

This application claims priority to U.S. Provisional Patent Application Ser. No. 63/643,954, filed May 8, 2024, the disclosure of which is incorporated herein by reference.

### BACKGROUND

The bone remodeling process is critical in maintaining homeostasis within the body; however, an imbalance in this cycle can lead to many life-altering disorders, including osteoporosis and Paget's disease. Skeletal homeostasis is maintained through the balance between the following bone cells: (i) osteoblasts that are responsible for new bone formation, (ii) multinucleated osteoclasts derived from hematopoietic precursors that are responsible for resorption activities, and (iii) osteocytes embedded within bone with mechano-signaling functions. Thus, distortion in the balance between the production and function of these critical bone cells is a prominent underlining feature of most bone disorders. The development of therapeutic techniques and drugs to treat such conditions has long been of interest to researchers and medical personnel, with compounds such as gallium being thoroughly investigated in recent years (Khosla S and Hofbauer LC, Lancet Diabetes Endocrinol. 2017 November; 5(11):898-907). Gallium compounds, specifically gallium nitrate, have been reported to exhibit dose-dependent downregulation against transcription factors such as NFAT2, TRAP, and c-Fos, which are responsible for osteoclast differentiation and subsequent bone resorption (Verron E et al., Drug Discov Today. 2012 October; 17(19-20):1127-32). Additionally, investigations using gallium compounds as potential treatments for cancer-related bone metastasis and hypercalcemia have shown reduced bone resorption both pre-clinically and clinically (Wang Y et al., Eur J Pharmacol. 2020 Dec. 15; 889:173613).

The inventors previously showed that a novel gallium compound, gallium acetylacetonate (GaAcAc), effectively inhibited osteoclast differentiation and function (Ghanta P. et al., Drug Deliv Transl Res. 2023 October; 13(10):2533-2549). They also demonstrated that application of gallium compounds without delivery systems could lead to diffusion out of the site of action to off-target sites, leading to either a higher dosage or a multiple-dosing regimen being needed, which increases the risks for adverse side effects. As a result, suitable drug delivery platforms are needed to maximize therapeutic efficacy at minimum effective doses (Sun W. et al., Mater Design. 2023 March 227:111704). Recent studies investigating the treatment of bone resorptive disorders such as osteoporosis and Paget's disease have focused on incorporating gallium compounds into medical implants and bioceramics to provide a localized effect and mitigate adverse reactions (He F. et al. Chem Eng J. 2021 May 412:128709).

Three-dimensional (3D) printing technology has become an area of interest in biomedical fields due to the high degree of customization, making it ideal for bone tissue engineering. We particularly focused on 3D-printing technology that applies fused deposition modeling whereby a polymer filament is melted (at high temperatures) and later printed layer-by-layer according to pre-determined specifications. A wide variety of filament materials and customizable software designs allow for modifications of the 3D printed materials to suit an individual drug's profile and increase its desired effects (Mohapatra S. et al., Sensors Int. 2022 3:100146). For bone regeneration, 3D-printed scaffolds have created an avenue for controlled drug release by encapsulating the drug of choice and slowly releasing it over time, providing a localized effect with increased benefit compared to the drug alone (Liu X. et al., Biomaterials. 2021 September; 276:121037). One potential challenge of 3D printed materials is loading the drug into the scaffold in a uniform manner. Multiple factors influence the even loading of the drug, including the heat being produced during printing and the shape of the scaffold (Calori IR. et al. Eur Polym J. 2020 April 129:109621). If loaded directly into the filament, the drug must withstand the heat generated to melt the filament during extrusion printing. If loaded after printing, it should be ensured that the drug is evenly coated on the scaffold at the intended concentration. It is imperative to confirm via characterization studies that, no matter the loading method used, the drug is evenly distributed throughout the scaffold, and the release of the drug occurs at the desired rate (Goole J. and A mighi K. Int J Pharm. 2016 Feb. 29; 499(1-2):376-394).

A second potential challenge for 3D printing biomaterials is using a filament material that is conducive to the specific drug and will produce non-toxic components upon degradation. This work used polylactic acid (PLA)-based 3D printed scaffolds. Polylactic acid is a naturally derived filament material widely used in 3D printed bone tissue engineering due to its non-toxic degradation and enhanced molecular adhesive properties. PLA can be easily modified by adding co-polymers and chemical coatings to maximize its benefits and overcome limitations such as hydrophobicity and biological inertness (Liu S. et al. Comp B Eng. 2020 October 199:108238.). One such coating is polydopamine (PDA), which has been previously studied for its ability to bind to proteins, nanoparticles, and genetic materials, as well as its osteogenic effect on cells (Chakka LRJ et al., Trans AMMM. 2020 Sep. 2(1)). As a result of its binding properties, PDA has been used extensively to increase the drug-loading capabilities of 3D printed scaffolds (Chakka JL et al., RSC Adv. 2021 Apr. 11 (22):13282-13291). A different treatment includes alkaline hydrolysis using sodium hydroxide (NaOH) to increase cell biocompatibility and drug loading. Using this method, carboxylate groups are introduced while chemical scission exposes hydroxyl and carboxylic acid groups on the surface, increasing surface roughness and decreasing hydrophobicity (Park S. et al., Polymers (Basel). 2021 Jan. 14; 13(2):257). The exposure of carboxylic acid groups creates a negative charge across the surface of the scaffold, increasing ionic exchange between the scaffold and a positively charged drug, thereby increasing the drug binding capabilities (Maia-Pinto MOC et al., Polymers (Basel). 2020 Dec. 27; 13(1):74).

### SUMMARY OF THE INVENTION

The inventors prepared 3D-printed polylactic acid scaffolds that were loaded with GaAcAc and investigated the impact of scaffold pretreatment with polydopamine (PDA) or sodium hydroxide (NaOH). They observed a remarkable increase in scaffold hydrophilicity with PDA or NaOH pretreatment while biocompatibility and in vitro degradation were not affected. NaOH-pretreated scaffolds showed the highest amount of GaAcAc loading when compared to other scaffolds (p<0.05). NaOH-pretreated scaffolds with GaAcAc loading showed effective reduction of osteoclast counts and size. The trend was supported by suppression of key osteoclast differentiation markers such as NFAT2, c-Fos, TRAF6, & TRAP. All GaAcAc-loaded scaffolds, regardless of surface pretreatment, were effective in inhibiting osteoclast function as evidenced by reduction in the number of resorptive pits in bovine cortical bone slices (p<0.01). The suppression of osteoclast function according to the type of scaffold followed the ranking: GaAcAc loading without surface pretreatment>GaA cAc loading with NaOH pretreatment>GaA cAc loading with PDA pretreatment.

### BRIEF DESCRIPTION OF THE FIGURES

The present invention may be more readily understood by reference to the following figures, wherein:

FIGS. 1A-1C provide graphs and images of 3D printing and surface pretreatment of PLA scaffolds for loading GaAcAc. (A) Illustration of 3D-printing process and scaffold surface pretreatment and separation into 3 groups: Blank (no surface pretreatment), PDA (treated with dopamine-Tris HCl solution), and NaOH (treated with alkaline solution). (B) Representative micro-CT images of blank 3D-printed PLA scaffolds (2 mm and 4 mm diameter). (C) Extent of GaAcAc loading onto different 3D scaffolds as measured by ICP analysis. Each datapoint represents mean±SD, n=3, *p<0.05. Schematic illustrations were made using BioRender software.

FIG. 2 provides an image showing the FTIR characterization of 3D-printed PLA scaffolds with or without GaAcAc loading. Scaffolds without GaAcAc loading (blank, PDA and NaOH) were characterized by FTIR as well as scaffolds loaded with GaAcAc (Blank-G scaffold, PDA-G-scaffold) and NaOH-G-scaffold). Characteristics of PLA are found in the blank scaffold (black arrows) while a shift in an alkane group was observed in the NaOH scaffold (red square). GaA cAc-loaded scaffolds all exhibited characteristics of acetylacetonate ligand of GaA cAc between 2900 and 3400 cm⁻¹ (green circle).

FIGS. 3A & 3B provide graphs and images showing the biocompatibility assessment of 3D-printed PLA scaffolds with or without GaAcAc loading. (A) Cell viability of RAW 264.7 and M C3T3 cells cultured with blank, PDA, and NaOH scaffolds for 24, 48, and 72 h. Data points represent mean±SD, n=3. * p<0.05 vs positive control (untreated control). (B) Live/dead staining for RAW 264.7 and MC3T3 cells plated on top of different 3D-printed PLA scaffolds and incubated (37° C.) for 24, 48 and 72 h.

FIGS. 4A-4D provide graphs and images showing the in vitro degradation of 3D-printed PLA scaffolds. (A). pH measurements of aliquots obtained from in vitro degradation studies of different scaffolds incubated in PBS at (A) 25° C. or (B) 37° C. for 60 days. (C) Swelling rate of different scaffolds for degraded scaffolds on day 60 of the in vitro degradation studies. (D) Macroscopic images of scaffolds incubated in PBS at 25° C. and 37° C. on day 60 of the in vitro degradation studies. Each datapoint represents mean±SD, n=3.

FIG. 5 provides a graph showing the biocompatibility assessment of 3D-printed PLA scaffolds after in vitro degradation. Scaffolds were submersed in PBS (37° C.) and monitored for 60 days. A fterwards, aliquots from scaffold degradation were mixed with cell culture media (1:5, aliquot: media) to assess cell viability in (A) RAW 264.7 and (B) M C3T3 cells for 24, 48, and 72 h. Each datapoint represents mean±SD, n=3; *p<0.05 vs. positive control.

FIGS. 6A-6C provide graphs and images showing the effects of GaAcAc-loaded 3D-printed PLA scaffolds on osteoclast differentiation. The effect of GaAcAc-loaded scaffolds (Blank-G, PDA-G and NaOH-G) versus unloaded scaffolds (blank, PDA, and NaOH) on osteoclasts differentiated with 30 ng/ml RANKL as assessed via (A) TRAP-staining, (B) the number of multinucleated TRAP-stained osteoclasts (black arrows), and (C) the size of the osteoclasts via differential count. Each datapoint represents mean±SD, n=4. *p<0.05, **p<0.01, ***p<0.001, ****p<0.0001, as compared to RANKL osteoclasts with 3-10 nuclei, bcompared to RANKL osteoclasts with ≥10 nuclei.

FIG. 7 provides an image showing the Western blot analysis of osteoclast differentiation. The effect of GaAcAc-loaded scaffolds (Blank-G, PDA-G and NaOH-G) versus unloaded scaffolds (blank, PDA, and NaOH) on osteoclasts differentiated with 30 ng/ml RANKL was assessed via western blot by the quantitative expression of osteoclast differentiation markers (A) NFAT2, (B) c-fos (C) TRAF6, and (D) TRAP normalized with GAPDH. Qualitative western blot images of all markers are shown in E. Datapoints represent mean±SD, n=4. *p<0.05, **p<0.01, ***p<0.001, ****p<0.0001 compared to positive control (RANKL).

FIGS. 8A & 8B provide graphs and images showing the effects of GaAcAc-loaded 3D-printed PLA scaffolds on osteoclast function. The effect of GaAcAc-loaded scaffolds (Blank-G, PDA-G and NaOH-G) versus unloaded scaffolds (blank, PDA, and NaOH) on osteoclast function via pit resorption assay using bovine bone slices. The resorptive pits were (A) examined toluidine blue stained to (B) assess number of resorptive pits. Each datapoint represents mean±SD, n=3; *p<0.05, **p<0.01 vs. positive control.

### DETAILED DESCRIPTION OF THE INVENTION

The present invention provides a biocompatible 3D-printed scaffold. The scaffold includes a biocompatible polymer shaped to form a scaffold using 3D printing and a gallium compound. Methods of making 3D-printed scaffolds, and methods of using 3D-printed scaffolds to inhibit bone resorption are also provided.

### Definitions

As used herein, the terms “treatment”, “treating”, and the like, refer to obtaining a desired pharmacologic or physiologic effect. The effect may be therapeutic in terms of a partial or complete cure for a disease or an adverse effect attributable to the disease. “Treatment”, as used herein, covers any treatment of a disease in a mammal, particularly in a human, and can include inhibiting the disease or condition, i.e., arresting its development; and relieving the disease, i.e., causing regression of the disease.

Prevention, as used herein, refers to treatment of a subject identified as being at risk of being afflicted with a condition or disease such as osteogenesis imperfecta, including avoidance of development of a bone disease or disorder, or a decrease of one or more symptoms of the bone disease or disorder should a bone disease or disorder develop nonetheless.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of an agent which will achieve the goal of improvement in disease severity and the frequency of incidence. The effectiveness of treatment may be measured by evaluating a reduction in symptoms in a subject in response to contact with the gallium-including scaffolds described herein.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

“Contacting,” as used herein, refers to causing two items to become physically adjacent and in contact, or placing them in an environment where such contact will occur within a short timeframe. For example, contacting a site with a scaffold comprising a gallium compound includes administering the composition to s subject at or near a site such that the gallium compound will interact with the site to inhibit bone resorption. In some embodiments, the step of contacting the site comprises surgically implanting the composition.

“Biocompatible” as used herein, refers to any material that does not cause injury or death to a subject or induce an adverse reaction in a subject when placed in contact with the subject's tissues. Adverse reactions include for example inflammation, infection, and cell death. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

The term “biodegradable” as used herein refers to a polymer that can be broken down by either chemical or physical process, upon interaction with the physiological environment subsequent to administration, and erodes or dissolves within a period of time, typically within days, weeks, or months. A biodegradable material serves a temporary function in the body, and is then degraded or broken into components that are metabolizable or excretable.

As used herein and in the appended claims, the singular forms “a”, “and”, and “the” include plural referents unless the context clearly dictates otherwise. Thus, for example, reference to “a compound” also includes a plurality of such compounds.

Unless defined otherwise, all technical and scientific terms used herein have the same meaning as commonly understood by one of ordinary skill in the art to which this invention belongs.

### Biocompatible 3D-printed Scaffold

In one aspect of the present invention, a biocompatible 3D-printed scaffold is provided that includes a biocompatible polymer shaped to form a scaffold using 3D printing and a gallium compound.

A variety of biocompatible polymers are known for use in 3D printing. Polymers for 3D printing are typically thermoplastic polymers. Examples of thermoplastic biocompatible polymers include polylactic acid, polycaprolactone, and polycarbonate, acrylonitrile, polyethylene teraphthalate glycol, polyamides, silicone, epoxy, and rigid polyurethanes. In some embodiments, the biocompatible polymers include polylactic acid, polycaprolactone, and polycarbonate. See Sta Agueda et al., MRS Commun., 11(2):197-212 (2021), the disclosure of which is incorporated herein by reference. The biocompatible polymer can be provided and used in various forms, such as filaments and pellets.

In some embodiments, the biocompatible polymer is polylactic acid. Polylactic acid is a thermoplastic polyester that is obtained by condensation of lactic acid or ring-opening polymerization of lactide. Polylactic acid includes racemic poly-|-lactic acid, regular poly-I-lactic acid, poly-d-lactic acid, and poly-dl-lactic acid. The polylactic acid composition may have an average molecular weight of 50,000 or more, preferably about 50,000 to 200,000, and more preferably about 50,000 to 150,000.

The polylactic acid monomer may be obtained by a conventional method for preparation of a polylactic acid homopolymer well known in the art. For instance, the monomer may be obtained by preparing L-lactide or D-lactide, a cyclic dimer, from L-lactic acid or D-lactic acid, respectively, and then conducting ring-opening polymerization of L-lactide or D-lactide, or by a direct polycondensation of L-lactic acid or D-lactic acid. Among those, the ring-opening polymerization is preferable to provide the polylactic acid repeat unit in a higher degree of polymerization. Further, the polylactic acid repeat unit may be formed by copolymerizing L-lactide and D-lactide at a certain ratio so as to render the copolymer non-crystalline. In order to further enhance the heat resistance of the polylactic acid resin composition, however, the polylactic acid repeat unit is preferably prepared by homopolymerization of either L-lactide or D-lactide.

In some embodiments, a polylactic acid copolymer is used. The polylactic acid copolymer can, for example, include polyether-based monomers. The polyether-based polyol monomers may be a polyether polyol (co)polymer prepared by ring-opening (co)polymerization of, e.g., one or more alkylene oxide monomers. Examples of the alkylene oxide include ethylene oxide, propylene oxide, butylene oxide, tetrahydrofuran, and the like. Exemplary polyether-based polyol monomers prepared from the alkylene oxide is at least one selected from the group consisting of a polyethyleneglycol (PEG) repeat unit, a poly(1,2-propyleneglycol) monomers, a poly(1,3-propanediol) monomers, a polytetramethyleneglycol monomers, a polybutyleneglycol monomers, a repeat unit of a polyol formed by copolymerization of propylene oxide and tetrahydrofurane, a repeat unit of a polyol formed by copolymerization of ethylene oxide and tetrahydrofurane, and a repeat unit of a polyol formed by copolymerization of ethylene oxide and propylene oxide. Preferably the polylactic acid copolymer comprises about 65 to 95% by weight ('wt. %) of polylactic acid and about 5 to 35 wt. % of polyether, based on the total weight (i.e., weight of the block copolymer).

Gallium compounds, as used herein, refers to gallium and gallium-including compounds, and in particular salts and coordination complexes of gallium. Preferably, the gallium compound is a non-radioactive gallium compound. In some embodiments, the gallium compound is selected from the group consisting of gallium acetylacetonate, gallium nitrate, and gallium citrate, gallium maltolate, gallium carbonate, gallium acetate, gallium triacetate, gallium tartrate, gallium oxide, gallium hydroxide, and gallium hydrated oxide. In further embodiments, the gallium compound is gallium acetylacetonate (Ga(acac)₃) which is a coordination complex of gallium having the formula Ga(C₅H₇O₂)₃. A therapeutically effective amount of the gallium compound should be included in the composition. In some embodiments, the gallium compound composition ranges from 5 mg/mL to 100 mg/ml of gallium compound in the scaffold material.

In some embodiments, the gallium compound has been loaded onto the surface of the 3D-printed scaffold. Gallium compound that has been loaded onto the surface of the scaffold is present only on the surface of the scaffold, and within a small region beneath the surface into which the gallium compound can diffuse from the surface of the scaffold. For example, in some embodiments, the gallium compound is present within only to a depth of about 0.1 mm to about 1 mm from the surface of the scaffold.

In some embodiments, the gallium compound is evenly loaded onto the surface of the 3D-printed scaffold, while in further embodiments the gallium compound is evenly distributed throughout the mass of the 3D printed scaffold. Gallium compound that has been evenly loaded onto the surface of the scaffold is present in a relatively constant concentration from one region of the surface of the scaffold to another. For example, in some embodiments, the concentration varies by less than 50%, by less than 25%, by less than 10%, or by less than 5% from one cm² region of the surface of the scaffold to another.

The composition for inhibiting bone resorption is configured as a tissue scaffold. A tissue scaffold is a support structure that provides a matrix for cells to guide the process of bone tissue formation in vivo. The morphology of the scaffold guides cell migration and cells are able to migrate into or over the scaffold, respectively. The cells then are able to proliferate and synthesize new tissue and form bone and/or cartilage.

The composition for inhibiting bone resorption can be molded or otherwise shaped during preparation to have any desired configuration as a tissue scaffold. Typically, the material is molded to have the shape of the bone or bone-like material that it is being substituted for. However, the scaffold material can also be used for cosmetic work or “bioengineering,” where a support structure is provided for the creation of new tissue rather than the replacement or regeneration of existing tissue. For further information regarding suitable tissue scaffolds for bone repair or regeneration, see for example U.S. patents applications Ser. Nos. 11/793,625, 12/193,794, 13/908,627, or 14/216,451, the disclosures of which are incorporated herein by reference.

In some embodiments, the scaffold is bioresorbable. Bioresorbable, as used herein, refers to the ability of the scaffolds to be gradually degraded by physiological processes in vivo, to allow the replacement of the biocompatible material with native tissue. For example, if the scaffold is used to replace bone, the scaffold may be gradually degraded while osteoblasts rebuild bone tissue in its place (i.e., bone remodeling).

In some embodiments, the biocompatible polymer composition further comprises any of various additives selected from the group consisting of an antioxidant, a reinforcing agent, and a combination thereof. For instance, the biocompatible polymer composition may further comprise an antioxidant (or a stabilizer) in order to prevent oxidation or thermal decomposition of the soft segment in a manufacturing process. The antioxidant may be at least one selected from the group consisting of a hindered phenol antioxidant, an amine antioxidant, a thio antioxidant, and a phosphate antioxidant. These antioxidants are well known in the art. The antioxidant used in the present invention may be present in an amount of 100 to 3,000 ppmw relative to the total weight of monomers used for forming repeat units of the polylactic acid composition.

The biocompatible polymer composition may further comprise a reinforcing agent to improve its anti-blocking property or the like. Examples of the reinforcing agent may include at least one selected from the group consisting of silica, colloidal silica, alumina, alumina sol, talc, mica, and calcium carbonate. Specific kinds or purchase routes of the reinforcing agent are well known to those skilled in the art.

Moreover, the biocompatible polymer composition may further comprise any other additives used in 3D printing, for example, plasticizers, UV stabilizers, anti-coloring agents, mat finishing agents, deodorizers, flame retardants, weather-proofing agents, antistatic agents, releasing agents, antioxidants, ion-exchangers, coloring pigments, inorganic or organic particles, or the like, as long as the composition is not adversely affected. Specific kinds or purchase routes of these additives are well known to those skilled in the art.

In some embodiments, the scaffold comprises a bone-seeking ligand. A bone-seeking ligand is a compound that can be included in the hydrogel that has an affinity for bone that encourages association of the hydrogel with bone. Examples of bone seeking ligands include alendronate, polyglutamic acid, and polyaspartic acid. See Wang et al., Bioconjugate Chemistry, 14(5):853-859 (2003). In some embodiments, the bone-seeking ligand is conjugated to the cellulose-based hydrogel.

In additional embodiments, the scaffold comprises a bone-retentive ligand. As used herein, a bone-retentive ligand refers to a material that can be conjugated and/or added to the biocompatible polymer to increase retention at the bone fusion site. The bone-retentive ligand helps to increase the retention of the drug at the bone fusion site and decrease further diffusion from the site of application. Examples of bone-retentive ligands are poly-aspartic acid, bisphosphonate, aspartic acid, glutamate, acidic oligopeptides, bisphosphonates, and alendronate.

### Methods of Making a Biocompatible 3D-printed Scaffold

A further aspect of the invention provides a method of making a biocompatible 3D-printed scaffold, comprising preparing a 3D-printed scaffold comprising a biocompatible polymer using a 3D printing method; and loading the surface of the 3D-printed scaffold with a gallium compound. The biocompatible 3D-printed scaffold can be any of the 3D-printed scaffolds described herein. For example, in some embodiments, the 3D-printed scaffold is a bone scaffold. In further embodiments, the gallium compound is gallium acetylacetonate, while in yet further embodiments the biocompatible polymer is polylactic acid.

Methods of preparing an object such as a scaffold using 3D printing are known to those skilled in the art. See Joseph et al., Int J Adv Manuf Technol., 125(3-4):1015-1035 (2023). Types of 3D printing suitable for use with biocompatible thermoplastic polymers include material extrusion, also referred to as fused deposition modeling (FDM), vat polymerization, powder bed fusion, material jetting, semi-solid extrusion, and binder jetting.

FDM may be the most common method of 3D printing, and the FDM process can be summarized as follows. The first step is to load a spool of thermoplastic filament into the printer. When the nozzle attained the desired temperature, the filament fed on the extrusion head and in the nozzle, the place where it melts. In some embodiments, the biocompatible polymer may have a melting temperature (Tm) of 170° C. or less, preferably about 145 to 170° C., and more preferably about 150 to 170° C. The extrusion head connected to a 3-axis system permits it to shift in X, Y, and Z directions. The melted material is extruded on thin strands and is stored layer-by-layer in preset positions, where it cools and solidifies. Sometimes the cooling of the material is expedited by cooling fans fitted on the extrusion head. In order to fill an area, it is necessary to have multiple passes, in a manner analogous to coloring a rectangle with a marker. When a layer is finished, the constructed platform goes up or down depending on the machine setups and another layer gets deposited. The action continues till the scaffold has been completed.

The method of preparing a biocompatible 3D printed scaffold includes the step of loading the surface of the 3D-printed scaffold with a gallium compound. For a description of methods of incorporating drugs into 3D printed materials, see Melnyk L. and Oyewumi, M., Annals of 3D printed medicine, v. 4, 100035 (2021). The gallium compound can be loaded onto the surface of the 3D printed scaffold using any suitable method of applying drugs to a polymeric surface, such as by spraying the gallium compound onto the surface, or immersing the 3D printed scaffold into a solution of the gallium compound for a suitable period of time (e.g., 8-12 hours) at about room temperature. Preferably, the method results in the relatively even distribution of the gallium compound onto the surface of the scaffold.

In some embodiments, the surface of the 3D-printed scaffold is modified to improve the loading of the gallium compound onto the surface. Surface modification of the 3D printed scaffold can alter the surface of the biocompatible polymer to improve gallium compound loading and/or the overall biocompatibility of the scaffold. In some embodiments, is treated with polydopamine or sodium hydroxide before loading the surface of the 3D-printed scaffold with a gallium compound. Treatment can be carried out by, for example, submerging the 3D printed scaffold in a dopamine hydrochloride or sodium hydroxide solution.

In some embodiments the gallium compound is loaded into the biocompatible polymer before forming the 3D printed scaffold. For example, gallium compound can be added to the biocompatible polymer before forming a 3D printed scaffold using semi-solid extrusion or powder bed methods of 3D printing. Alternately, binder jetting 3D printing to create scaffolds loaded with gallium. In this process, there is a flow of liquid binding agents as droplet sprays from a nozzle onto a thin layer of powder formulation upon a build plate. An additional layer of powder reservoir is rolled in step by step throughout the printing process. The steps of application of binding solution and layering are repeated until all required layers are fused together. In this case, gallium compound can be included in the binder solution to provide uniform incorporation of small quantities of the gallium compound into the biocompatible polymer forming the 3D printed scaffolds. In such cases, the gallium compound may be evenly distributed throughout the 3D printed scaffold.

### Methods for Inhibiting Bone Resorption

In another aspect, a method of inhibiting bone resorption in a subject is provided. The method includes implanting a biocompatible 3D-printed scaffold comprising implanting a biocompatible 3D-printed scaffold comprising a biocompatible polymer shaped to form a scaffold using 3D printing and a therapeutically effective amount of a gallium compound into the subject. The biocompatible 3D-printed scaffold can include any of the gallium-loaded scaffolds described herein. For example, in some embodiments, the scaffold is a bone scaffold, while in further embodiments the gallium compound is gallium acetylacetonate, and in yet further embodiments the biocompatible polymer is polylactic acid.

Bone resorption is the process by which osteoclasts break down the tissue in bone, releasing the minerals and resulting in a transfer of calcium from bone tissue to the blood. While bone resorption is generally a healthy process involved in routine bone remodelling, in some cases it can be helpful to inhibit bone resorption, resulting in a decreased rate of bone tissue breakdown.

In some embodiments the biocompatible 3D-printed scaffold stimulates osteoclast differentiation. Osteoclast differentiation is the process by which osteoclast progenitor cells develop into mature osteoclasts, which are responsible for bone resorption. This process involves several key stages, including commitment, maturation, and fusion, and is tightly regulated by cytokines like macrophage colony-stimulating factor (M-CSF) and receptor activator of nuclear factor-KB ligand (RANKL).

A change in the level of bone resorption can readily be determined by comparing levels in a subject before and after treatment. The level of bone resorption and/or bone mass before and/or after treatment may be determined from a series of measurements taken over different timepoints to provide a standard range. The level of bone resorption and/or bone mass before and/or after treatment may be measured in multiple individuals to provide a standard range representative of a given population. In certain embodiments, the level of bene resorption may be decreased in a subject treated by the methods of the present invention by at least about 2%, at least about 3%, at least about 4%, at least about 5%, at least about 10%, at least about 15%, at least about 20%, at least about 30%, at least about 40%, at least about 50%, at least about 60%, at least about 70%, or at least about 80%, compared to the level of bone resorption prior to treatment.

A bone (i.e., bone tissue) is a rigid organ that constitutes part of the vertebral skeleton. Bone tissue includes two basic types-cortical (the hard, outer layer of bone) and cancellous bone (the interior trabecular or spongy bone tissue), which gives it rigidity and a coral-like three-dimensional internal structure. Other types of tissue found in bone include marrow, endosteum, periosteum, nerves, blood vessels and cartilage. Bone is an active tissue composed of different cells. Osteoblasts are involved in the creation and mineralization of bone; osteocytes and osteoclasts are involved in the reabsorption of bone tissue. The mineralized matrix of bone tissue has an organic component mainly of collagen and an inorganic component of bone mineral made up of various salts.

The present invention can be used to decrease the rate of bone resorption any type of bone. There are five types of bones in the human body. These are long bones, short bones, flat bones, irregular bones and sesmoid bones. Examples of long bones include the femur, the humerus and the tibia. Examples of short bones include carpals and tarsals in the wrist and foot. Examples of flat bones include the scapula, the sternum, the cranium, the os coxae, the pelvis, and ribs. Irregular bones are those which do not fit within the other categories, and include vertebrae, sacrum and mandible bones. Sesmoid bones are typically short or irregular bones, imbedded in a tendon, such as the patella. While not formally considered bone, teeth are also included in the definition of bone used herein.

Bone injury can occur as a result of disease, chronic stress, or physical trauma. Examples of different types of bone injury include degenerative disc, cervical spondylosis, and bone fracture. Bone regeneration is also called remodeling and occurs at the cellular level. When the process becomes unbalanced, e.g., from too much resorption, bone mass decreases and bones may become brittle. Decreasing the rate of bone resorption that occurs over a given time can be used to increase bone repair. For example, enhancing bone repair includes decreasing the rate or amount of bone resorption by up to 10%, 20%, 30%, 40%, 50%, 60%, 70%, 80%, 90%, or even 100% compared with the amount or rate of bone resorption that would occur in an untreated subject.

In some embodiments, the subject has been diagnosed as having a bone growth disease or disorder, or suspected to be suffering from a bone growth disease or disorder. A Iternatively, subjects treated may not be suffering from a bone growth disease or disorder, but may be susceptible (i.e., have a higher risk of developing) to a bone growth disease or disorder. For example, a subject susceptible to suffering from a bone growth disease or disorder can be a subject that has been diagnosed as having an increased risk of developing a bone growth disease or disorder. A determination of whether a given subject is suffering from a given bone growth disease or is susceptible to a given bone growth disease can be made by those of skilled in the art based on clinical symptoms and/or other standard diagnostic tests which may vary depending on the particular disease in question.

As used herein, “bone growth disease” or “bone growth disorder” refers to a disease or condition associated with abnormality of the bone that can be treated by increasing bone mass and/or bone growth. A wide variety of bone growth disease and disorders are known to those skilled in the art. Examples of bone growth diseases and disorders include, but are not limited to: Achondrogenesis; Achondroplasia; Acrodysostosis; Acromesomelic Dysplasia (Acromesomelic Dysplasia Maroteaux Type, AMDM); Atelosteogenesis; Campomelic Dysplasia; Cartilage Hair Hypoplasia (CHH) (Metaphyseal Chondrodysplasia, McKusick type); Chondrodysplasia Punctata; Cleidocranial Dysostosis; Conradi-Hunermann Syndrome; Cornelia de Lange; Cranioectodermal dysplasia; Desbuquois syndrome; Diastrophic Dysplasia; Dyggve-Melchior-Clausen; Dyssegmental Dysplasia; Ellis van Creveld Syndrome (Chondroectodermal Dysplasia, EVC); Growth Hormone Deficiency; Hallerman-Streiff Syndrome; Hunter Syndrome (MPS II); Hurler-Scheie Syndrome (MPS I); Hypochondrogenesis; Hypochondroplasia; Hypophosphatasia; Hypophosphatemia; Hypopituitary; Hypothyroidism; Jarcho-Levin Syndrome (Spondylothoracic Dysplasia, Spondylocostal); Jeune Syndrome (Asphyxiating Thoracic Dysplasia; Asphyxiating Thoracic Dystrophy); Kniest Dysplasia; Laron Dwarfism; Larsen Syndrome; Leri-Weill Dyschondrosteosis (Mesomelic Dwarfism, Madelung Deformity); Lethal Skeletal Dysplasias; Maroteaux-Lamy (MPS VI) (MPS VI); Mesomelic Dysplasia; Metaphyseal Chondrodysplasia-Jansen Type; Metaphyseal Dysplasia-Schmid Type; Metatropic Dysplasia; Morquio Syndrome (MPS IV); Mucopolysaccharidoses; Multiple Epiphyseal Dysplasia (MED); Osteogenesis Imperfecta (OI); Pituitary Dwarfism; Precocious Puberty; Primordial Dwarfism (Microcephalic Osteodysplastic Primordial Dwarfism, MOPD); Pseudoachondroplasia; Rhizomelic Chondrodysplasia Punctata; Rickets; Robinow dwarfism/syndrome; Russell-Silver Syndrome; SADDAN: Severe Achondroplasia with Acanthosis Nigricans and Developmental Delay; Schmike Immuneosseous Dysplasia; Seckel Syndrome; Short Rib Polydactyly; Shwachman-Diamond Syndrome; Spondyloepimetaphyseal Dysplasia-Strudwick (SEMD); Spondyloepimetaphyseal dysplasias; Spondyloepiphyseal Dysplasia; Spondyloepiphyseal Dysplasia Congenita (SED-Congenita, SEDC); Spondyloepiphyseal Dysplasia Tarda (SED-Tarda, SEDT, SED-L); Spondylometaphyseal Dysplasia-Corner Fracture Type (SMD, SMD-Corner Fracture Type, SMD-Sutcliffe Type); Spondylometaphyseal Dysplasia-Kozlowski (SMD-Kozlowski, SMDK); Thanatophoric Dysplasia; Trichorhinophalangeal Syndrome (Langer-Giedion syndrome); Turner Syndrome; Type II Collagenopathies.

In some embodiments, the subject has been diagnosed as having bone growth disease or disorder selected from the group consisting of osteogenesis imperfecta, disorders caused by increased osteoclastogenesis or bone loss associated with inflammatory conditions, infection, genetic and age-related bone disorders such as osteoporosis, osteopenia, Paget's disease, metastatic bone cancer, myeloma bone disease, bone fracture healing, and bone graft repair.

Bone disease or disorders characterized by a loss of bone mass also encompass abnormalities in the strength and structures of the bones. Examples include, but are not limited to, decreased bone mass, change in bone density, bone softness, tumors on bones and abnormal bone architecture. Additionally or alternatively, a diagnosis of a given bone disease can be made by a physician, nurse, or veterinarian, depending on the subject under consideration.

The level of or changes in bone mass can be useful to determine if a subject is suffering from a bone growth disease or disorder, or to evaluate treatment of a bone growth disease or disorder by decreasing bone resorption. Bone mass may be measured in mammalian subjects (e.g. humans) using standard techniques (e.g. dual energy X-ray absorptiometry (DXA) ) . In particular, DXA may be used for diagnosis, prognosis (e.g. fracture prediction), monitoring the progression of a bone disease, and/or assessing responses to treatment. Categorization of subjects into diseased and non-diseased states based on bone mass (i.e. bone material density) can be made on the basis of standard classification systems including those published by the World Health Organization (see, for example, World Health Organization Technical Report Series 921 (2003), Prevention and Management of Osteoporosis). For example, a diagnosis of osteoporosis may be based on BMD that is two standard deviations or more below a young adult reference mean.

The composition used to inhibit bone resorption can include any of the compositions described herein. In some embodiments, the gallium compound is selected from the group consisting of gallium acetylacetonate, gallium nitrate, and gallium citrate. In further embodiments, the gallium compound of the composition is gallium acetylacetonate.

In some embodiments, the gallium compound is released from the 3D-printed polylactic acid scaffold in a sustained release manner. A variety of release kinetics are contemplated for the timed release of drug(s) from the scaffold, including bi-or multi-phase release, such as an initial fast release followed by a slower subsequent release phase or delay the initial release for a certain period of time and then rapid or sustained release. For example, the release may include a burst release of drug from the scaffold rapidly within seconds or minutes followed by further sustained release over a period of at least 2, 4, 6, 8 or more hours, days, weeks and/or months. Such longer-term release can be referred to as sustained or prolonged release. Such release kinetics may be advantageous in certain circumstances, e.g., where sustained action is desired.

In some embodiments, the method of inhibiting bone resorption also includes administration of one or more additional bone growth stimulating agents. The term “bone growth-stimulating agent” is intended to include any material that stimulates and encourages the development and functional maintenance of bone, and mature osteoclasts in bone. Examples of bone growth-stimulating agents include, without limitation: growth factors; cytokines and the like, such as members of the Transforming Growth Factor-beta (TGF-□) protein superfamily including any of the Bone Morphogenic Proteins (BMP) and members of Glycosylphosphatidylinositol-anchored (GPI-anchored) signaling proteins including members of the Repulsive Guidance Molecule (RGM) protein family; other growth regulatory proteins, and bisphosphonates.

Because patients with a bone disease or disorder have heterogeneous clinical manifestations and varying clinical outcomes, the treatment given to a subject may vary, depending on their prognosis. The skilled clinician will be able to readily determine without undue experimentation specific secondary agents, types of therapy that can be effectively used to treat an individual subject with a bone disease or disorder.

The method for inhibiting bone resorption includes in vivo placement of a scaffold for inhibiting bone resorption, as described herein for bioengineering, restoring or regenerating bone. In particular aspects of the method, bioengineering, restoring or regenerating bone is in vitro or ex vivo, including placement under body fluid conditions. The method includes positioning the scaffold for inhibiting bone resorption to provide structural support for nearby tissue. In some embodiments of the method, the compositions are used for dental and orthopedic implants, craniomaxillofacial applications and spinal grafting, and said composition is suitable to promote bone regeneration and repair.

Preferably the method of inhibiting bone resorption occurs under aseptic conditions. “Aseptic” as the term is used herein, refers to methods to control or reduce the microbial bioburden in an environment. Tissues processed “aseptically” are tissues processed using sterile instruments, and special environmental surroundings (including for example “clean room technologies”).

In further embodiments, the site in need of decreased bone resorption is a dental site. Decreasing bone at a dental site can improve the repair of teeth, or bone tissue near the teeth. The bone resorption inhibiting composition can be used as part of a bone repair process following extraction of a tooth and/or placement of a dental prosthesis, or for repairing dental bone defects such as bone loss from moderate or severe periodontitis.

Examples have been included to more clearly describe a particular embodiment of the invention and its associated cost and operational advantages. However, there are a wide variety of other embodiments within the scope of the present invention, which should not be limited to the particular examples provided herein.

### EXAMPLES

### Example 1: Fabrication of 3D-Printed Scaffolds Loaded with Gallium Acetylacetonate for Application in Osteoclastic Bone Resorption

The main hypothesis of our work is that GaAcAc can be loaded successfully onto 3D-printed PLA scaffolds for potential application in resorptive disorders through inhibition of osteoclast activity. Key objectives for this work are (a) to develop and characterize 3D printed PLA scaffolds by fused-deposition modeling approach that will include post-manufacture loading process for GaAcAc, and (b) to evaluate the impact of scaffold surface pretreatment with PDA or NaOH prior to GaAcAc loading. All scaffolds were characterized based on weight, pore size, hydrophilicity, in vitro degradation (60 days), biocompatibility with bone cells, osteoclast differentiation and function using the extent of resorption (ex-vivo) in cortical bovine bone slices.

### Materials and methods

### Materials

Tris was purchased from VWR (Randor, PA). Sodium hydroxide (NaOH) was obtained from J.T. Baker (Randor, PA). 4× laemmli buffer, Trans-Blot Turbo Mini-size Transfer Stacks, and 10× Transfer buffer were obtained from Bio-Rad (Hercules, CA). Fast Violet Red Ib Salt Zinc Chloride was purchased from MedChem Express (Monmouth Junction, NJ). Trypsin, phosphate buffered saline (PBS), Dimethyl sulfoxide (DMSO), and p-nitrophenyl phosphate were purchased from Fisher Scientific (Pittsburgh, PA). ImmobilonVR Forte Western HRP substrate was procured from Millipore (Burlington, MA). Gallium acetylacetonate (GaAcAc) was purchased from STREM Chemicals (Newburyport, MA). All other materials, unless otherwise stated, were purchased from Sigma-Aldrich (St. Louis, MO).

### PLA Scaffold Printing and Surface Pretreatment

Scaffolds were printed using polylactic acid filament of 1.75 mm thickness (Prusa Research) on the Original Prusa i3 M K 3S+printer (Prusa Research, Prague, Czech Republic). Using Fusion 360 software (Autodesk, USA), two models of the scaffold were prepared with dimensions of 2.00×4.50 mm and 4.00×4.50 mm. Printing conditions used for the scaffolds are as follows: layer height: 0.05 mm, print speed: 20 mm s⁻¹, nozzle temperature: 210° C., bed temperature: 50° C., and 70% infill density (Karanth D. et al., Clin Exp Dent Res. 2023 Apr; 9(2):398-408). The printed scaffolds were stored at room temperature.

The surface of the 2 mm scaffolds was modified using polydopamine (PDA) or alkaline hydrolysis (NaOH). For the PDA surface pretreatment, the scaffolds were submerged (24 h) in dopamine hydrochloride solution (2 mg/ml; 2 ml/scaffold) with 10 mM Tris buffer under gentle agitation. Subsequently, the scaffolds were air dried for a minimum of 15 h and stored at room temperature for further use (Chakka LRJ et al., Trans AMMM. 2020 Sep. 2 (1)).

Similarly, to modify the scaffold via alkaline hydrolysis, the scaffolds were submersed in 0.2 N NaOH solution (2 ml/scaffold) for 1 h under gentle agitation then rinsed three times with water, once with 0.1 N hydrochloric acid, and again in water. All scaffolds were dried completely for a minimum of 15 h before use and stored at room temperature (Donate R. et al., Polymers (Basel). 2021 May 18; 13(10):1643). Scaffolds were termed according to types of surface pretreatments as Blank (for scaffolds without surface pretreatment), PDA (for scaffolds that had PDA surface pretreatment) and NaOH (for those that received NaOH surface pretreatment).

### Loading of GaAcAc onto Scaffolds

GaAcAc was loaded onto Blank scaffolds (with no surface pretreatment, Blank-G) and those that received PDA (PDA-G) or NaOH (NaOH-G) surface pretreatment. The scaffolds were submerged in a solution of GaAcAc (5 mg/ml) at 37° C. overnight and allowed to dry fully at room temperature. The amount of GaAcAc loaded onto the scaffolds was determined via inductively coupled plasma atomic emission spectroscopy (ICP), (Thermo Scientific iCAP 7400) using our earlier reported method (Ghanta P. et al., Drug Deliv Transl Res. 2023 October; 13(10):2533-2549) and validated using standard ICP gallium solution. Briefly, each scaffold was placed in 2 ml of nitric acid placed on a shaker for 20 h. The resultant suspension was filtered (0.45 mm).

### Characterization of Scaffolds

Macroscopic images of the scaffolds were obtained using a Nikon SM Z800 stereo microscope with a Nikon DS-Fi1 digital camera (Nikon, Minato City, Japan). Fourier Transform Infrared Spectrophotometer (FTIR) spectra were obtained using a Bruker FTIR spectrometer (Bruker Optics Inc., Manning Park, USA) on both GaAcAc-loaded (Blank-G, PDA-G, NaOH-G) and unloaded scaffolds in the scanning range of 500-4000 cm⁻¹ at room temperature. Scaffolds were characterized via weight, porosity, contact angle, and swelling rate.

### Porosity

To obtain the average porosity of scaffolds, we first calculated the volume (Vs) of each scaffold by measuring the edges of the scaffolds using vernier calipers (Bel-Art, Wayne, NJ). The weight of the scaffold (WS) was divided by the density of the filament (q-filament ¼ 1.24 g/cm3) to obtain the volume of the filament (VF). The ratio of the scaffold volume and volume of the filament was used to generate the porosity as adapted from an earlier study using the following equation (Tanaka Y. et al., Biomaterials. 2010 June; 31(16):4506-16).

$$\% Porosity = ( {V}_{S} - {V}_{F} ) / {V}_{S} \times 100 = {V}_{S} - ( {W}_{S} / p {-}_{filament} ) / {V}_{S} \times 100 \qquad ( 1 )$$

### Contact Angle

To measure the contact angle, scaffolds were placed on the stage of an Ossila Contact Angle Goniometer (Ossila, Sheffield, UK) and a drop of water (10 mL) was placed on top using the provided syringe (Park S. et al., Polymers (Basel). 2021 Jan. 14; 13(2):257). An image was captured and analyzed using Ossila Contact Angle Software (Ossila, Sheffield, UK).

### Swelling Rate

The swelling rate was measured by submerging the scaffolds in 3 ml of water for 24 h at 25° C. and 37° C. Prior to submersion, the dry weight (Wd) of the scaffolds was obtained. After 24 h, the wet weights (WS) of the scaffolds were measured, and the swelling rate was calculated using the following equation (15):

$$\% Swelling Rate \times [ ( {W}_{S} - {W}_{d} ) / {W}_{d} ] \times 100 \qquad ( 2 )$$

### Degradation

Blank and surface-modified scaffolds were placed into scintillation vials filled with 3 ml of either double-filtered water (H₂O scaffolds) or PBS (PBS scaffolds) and stored at three temperatures: 4° C., 25° C., and 37° C. for 60 days. At designated time points, the water or PBS was removed, measured for pH via pH meter (Mettler Toledo), and replaced with fresh liquid. This occurred a total of 16 times for the water and 10 times for the PBS groups. Both the dry and wet weights of the scaffolds were obtained on Day 0 and Day 60 and used to calculate the swelling rate using Equation 2 above. A Hitachi S-2600N scanning electron microscope (SEM—Hitachi High-Tech Corporation, Tokyo, Japan) was used to obtain images of the dried scaffolds at 30× and 100× magnification to assess any visual surface changes resulting from degradation. Macroscopic images of the scaffolds were also obtained to detect color changes. After drying, the porosity and contact angle were measured as stated above on the degraded scaffolds.

### In Vitro Studies

### Cell Culture

RAW 264.7 (pre-osteoclast) and MC3T3 (pre-osteoblast) cells (ATCC, Manassas, VA) were grown in Dulbecco's Modified Eagle's Medium (DMEM) and Alpha-modified eagle's medium (a-MEM) (Corning Inc., Corning, NY) respectively, supplemented with 10% fetal bovine serum (FBS-Millipore, Burlington, MA)) and 1% penicillin-streptomycin (PenStrep-Sigma-Aldrich, St. Louis, MO). Cells were housed in an incubator under humified conditions of 37° C. and 5% CO₂.

### Scaffold Biocompatibility and Cell Attachment

RAW 264.7 and MC3T3 cells were seeded at a density of 3,000 and 5,000 cells/well, respectively, in a 96-well plate and incubated overnight. Scaffolds were incubated in direct contact with cells for 24, 48, and 72 h. At the end of each respective time point, the scaffolds were removed, and the cells were treated with Alamar blue in media (prepared per manufacturer's instructions, GBiosciences, St. Louis, MO). Following 4 h of incubation, absorbance was measured using a SpectraMax UltraViolet Plate Reader (Molecular Devices LLC, San Jose, CA) at 570 nm and 600 nm. Viability was compared to untreated control cells that were not incubated with scaffolds.

In addition, cytotoxic effects of the scaffolds on cells were visualized using a LIVE/DEAD™ Viability/Cytotoxicity Kit (ThermoFisher Scientific, Waltham, MA). Briefly, RAW 264.7 and MC3T 3 cells were seeded on sterilized scaffolds at 50,000 cells/100 mL and incubated for 30 min for cell attachment, after which an additional 100 ml of media was added. After 24, 48, and 72 h of incubation, the scaffolds were rinsed with PBS and incubated with the live/dead working solution containing Calcein AM and BOBO-3 Iodide for 20 min at room temperature in the dark. Live cells (green) and dead cells (red) were imaged using an AxioZoom V.16 fluorescent microscope (Zeiss Group Global, Oberkochen, Germany).

### Degradation Biocompatibility

RAW 264.7 and MC3T3 cells were seeded at 3,000 cells/well and 5,000 cells/well, respectively, in 96-well plates and incubated overnight. Water obtained from Day 60 for scaffolds stored at 25° C. and 37° C. was sterilized and mixed with media at ratios of 1:5 and 1:10 before being added to the cells. Cells were incubated for 24, 48, and 72 h and viability was assessed using Alamar Blue. Viability was compared to untreated control RAW and MC3T3 cells that were treated with culture media.

### Osteoclast Differentiation

Adapting our earlier reported method for osteoclast differentiation (Ghanta P. et al., Drug Deliv Transl Res. 2023 October; 13(10):2533-2549), pre-osteoclastic cells (RAW 264.7 cells) were seeded at 5,000 cells/well in the bottom of a 24-well transwell plate (Corning Inc., Corning, NY). Following overnight incubation, both GaAcAc-loaded and unloaded scaffolds were placed in the top insert plate and cells were treated with differentiation media, i.e. DMEM supplemented with 30 ng/ml RANKL (R&D Technologies, Minneapolis, MN) every two days. On Day 5, the cells were fixed with 4% paraformaldehyde for 10 min at room temperature and evaluated for osteoclast differentiation via Tartrate-Resistant Acid Phosphatase (TRAP) staining. TRAP-positive cells (pink colored with ≥3 nuclei) were imaged and counted as differentiated osteoclasts via ImageJ. To assess the size of the osteoclasts, a differential count was performed in which osteoclasts were separated into two categories: those with ≥10 nuclei and those with <10 nuclei.

### Western Blot Analysis

To further assess the effects of the scaffolds on osteoclast differentiation, osteoclasts were seeded and differentiated as stated above until Day 5, when they were lysed with 1× RIPA buffer containing protease inhibitor (ThermoFisher Scientific, Waltham, MA). After centrifugation, the supernatants were analyzed via western blot for the following osteoclast differentiation markers: NFAT2, c-Fos, TRAF6, and TRAP (Cell Signaling Technology, Danvers, MA). Briefly, lysates were first analyzed via BCA Pierce Assay kit (ThermoFisher Scientific, Waltham, MA) to quantify the protein content. Then, protein samples were separated via 10% SDS-page gel, transferred onto a polyvinylidene fluoride (PV DF) membrane (Bio-Rad, Hercules, CA), and blocked with 5% BSA for one hour. After washing with 1× TBST, the membrane was probed with primary antibodies overnight, using GAPDH (Cell Signaling Technology, Danvers, MA) as a loading control, and incubated with a secondary HRP conjugated anti-rabbit IgG secondary antibody (Cell Signaling Technology, Danvers, MA) for one hour prior to imaging. Quantification of band expression was conducted using ImageJ software and all data was normalized via GAPDH protein expression.

### Pit Resorption Assay

Functionality of the differentiated osteoclasts was assessed via a pit resorption assay. Briefly, RAW cells were seeded at 7,500 cells/well onto bovine cortical bone slices (BioVendor, Asheville, NC) and differentiated in direct contact with drug-loaded and unloaded scaffolds as described above. On Day 5, the scaffolds were removed, and the bone slices were fixed with 4% paraformaldehyde. To visualize the differentiated osteoclasts, the bone slices were TRAP stained, imaged, and the differentiated osteoclasts were counted. The bone slices were then cleaned with 10% bleach via sonication for at least 15 min and stained with 0.5% toluidine blue (Sigma-Aldrich, St. Louis, MO) for three minutes at room temperature. After washing five times, the slices were imaged and the number of resorption pits were counted (Trebec DP et al., J Cell Biochem. 2007 May 1; 101(1):205-20).

### Data Analysis

All data is presented as Mean±SD. Statistical significance was evaluated using a student's t-test or ANOVA with Dunnett's or Tukey's multiple comparison post-hoc tests at a significance level of p<0.05. All graphs were prepared using GraphPad Prism 9 (GraphPad Software, CA).

### Results

### Characterization of Scaffolds

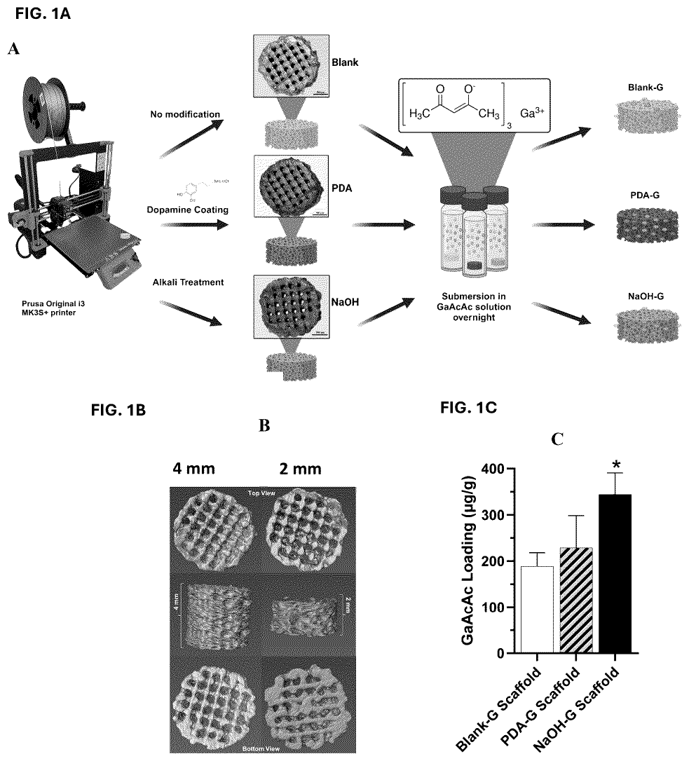

A schematic for 3D-printed PLA scaffolds is provided with an outline of different scaffold pretreatments before GaA cAc loading (FIG. 1A). Initial scaffold designs included 2 mm and 4 mm diameter models produced using the same 3D printing parameters. The micro-CT images for the 2 mm and 4 mm scaffolds are shown (FIG. 1B). For characterization, scaffolds were printed in quadruplicate, and the weight, swelling rate, and porosity were analyzed for consistency in replication (data not shown). Both scaffold diameter models exhibited consistent weights, similar swelling rates, and porosity measurements (data not shown). Ultimately, the 2 mm scaffold diameter was chosen for experimentation due to anticipation of shorter degradation time compared to 4 mm diameter scaffolds.

Blank scaffolds without any surface pretreatment and loaded with GaA cAc were called Blank-G (FIG. 1). Scaffolds treated with PDA before loading GaAcAc were classified as PDA-G while scaffolds that received NaOH pretreatment before GaAcAc loading were termed NaOH-G (FIG. 1). After surface pretreatment, all scaffolds were dried thoroughly and examined under a stereoscope for macroscopic changes, followed by assessment for differences in weight, contact angle, swelling rate, and porosity (Table 1). While no changes in scaffold weight or porosity were detected after surface pretreatments, we observed differences in the contact angle, an essential measurement for hydrophilicity (Table 1). Additionally, surface pretreatments caused an increase in swelling rate when stored in water overnight at 25° C. (data not shown); however, this trend was not repeated when stored at 37° C. (Table 1). After overnight submersion in the GaAcAc solution, ICP analysis revealed successful loading of GaAcAc onto the 3D-printed PLA scaffolds (FIG. 1C). GaAcAc loading onto PDA and Blank scaffolds were comparable, while NaOH scaffolds showed a slightly higher amount of GaA cAc loading (p<0.05; FIG. 1C).

TABLE 1
Characterization of 3D-printed PLA scaffolds at the time
of fabrication. Scaffolds that received surface pretreatments
(PDA scaffold and NaOH scaffold) were compared to scaffolds
without surface pretreatment (blank scaffold). Swelling
rate recorded for scaffolds stored at 37° C.

| | | | NaOH |
|---|---|---|---|
| Parameters | Blank Scaffold | PDA Scaffold | Scaffold |
| Average weight (mg) | 26.6 ± 0.26 | 26.6 ± 0.26 | 27.2 ± 1.0 |
| Contact angle (° C.) | 87.5 ± 7.75 | 49.9 ± 8.66** | 76.5 ± 9.95 |
| Porosity (%) | 99.93 | 99.94 | 99.93 |
| Swelling rate (%) | 36.65 ± 6.93 | 38.49 ± 1.93 | 34.83 ± 7.28 |

**p < 0.01 versus Blank scaffold. Each datapoint represents mean ± SD, n = 3 scaffolds.

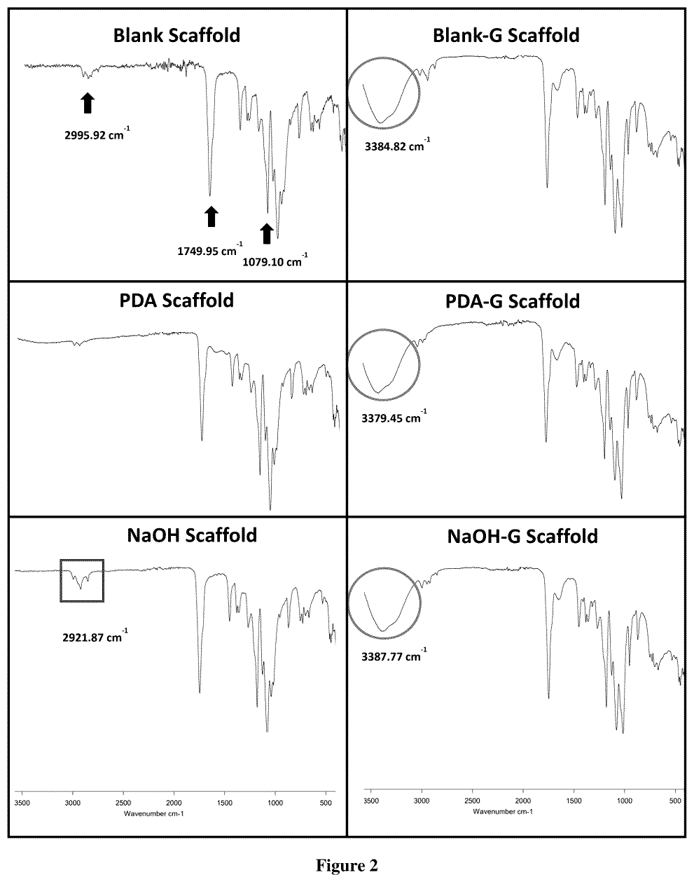

All scaffolds were further characterized by FTIR according to different types of surface pretreatments with or without GaA cAc loading (FIG. 2). The spectra of all scaffolds exhibited common characteristic stretching frequencies previously reported for PLA, including the presence of C═O, CH₃, and C—O at the following wavenumbers: 1748±1, 2993±3, and 1080 cm⁻¹, respectively (Jose J. et al., RSC Adv. 2020 Oct. 14; 10(62):37928-37937). The Blank scaffold (with no surface pretreatment) exhibited a C—H stretch at 2946.26 cm⁻¹, whereas upon surface treatment with NaOH, a shift in the wavenumber from 2946.26 to 2921.87 cm⁻¹ was observed. FTIR spectra of scaffolds loaded with GaAcAc all exhibited a broad O—H peak at 3384±5 cm⁻¹ and a C—H stretch peak at 2922 cm⁻¹ (FIG. 2).

### Biocompatibility and Cell Attachment to Scaffolds

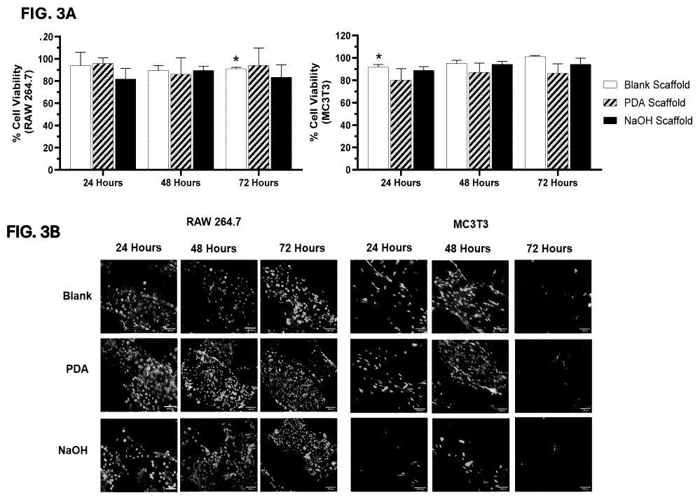

Biocompatibility of the scaffolds was assessed via direct contact with pre-osteoclastic RAW 264.7 and pre-osteoblast MC3T3 cells (FIG. 3). Scaffolds with surface pretreatments exhibited no significant reduction in viability on either cell line. We detected a slight reduction in cell viability for Blank scaffolds at 72 h (RAW) and 24 h (MC3T3) (p<0.05; FIG. 3A), however the effects are not pronounced.

Similarly, Live/Dead staining was used to analyze cell attachment and proliferation qualitatively. Both RAW 264.7 and MC3T3 cells successfully attached and remained viable on all three scaffold groups (FIG. 3B). High biocompatibility was determined based on the visual observation of live cells (green) and dead cells (red) (FIG. 3B). We also observed that the pre-osteoclastic RAW 264.7 cells showed higher prominence and attachment to the scaffolds when compared to MC3T3 cells (FIG. 3B). We observed a higher cell attachment to PDA-pretreated scaffolds than NaOH-pretreated ones.

### Degradation of Scaffolds

TABLE 2
Characterization of 3D-printed scaffolds on day 60 following in vitro
degradation studies. in vitro degradation was conducted by incubating
scaffolds at 25° C. or 37° C. with phosphate buffered saline.

| | Parameters | | | | | |
|---|---|---|---|---|---|---|
| | Blank Scaffold | | PDA Scaffold | | NaOH Scaffold | |
| Temp. Group | 25° C. | 37° C. | 25° C. | 37° C. | 25° C. | 37° C. |
| Average Weight | 27.1 ± 0.4 | 27.1 ± 0.2 | 27.5 ± 0.64 | 27.1 ± 0.2 | 27.0 ± 0.3 | 27.3 ± 0.20 |
| (mg) | | | | | | |
| Contact Angle | 84.0 ± 3.0 | 74.7 ± 9.9 | 51.3 ± 10.4** | 68.06 ± 14.7 | 79.03 ± 10.6 | 62.66 ± 5.13 |
| (°) | | | | | | |

**p < 0.01 versus Blank scaffold. Each datapoint represents mean ± SD, n = 3.

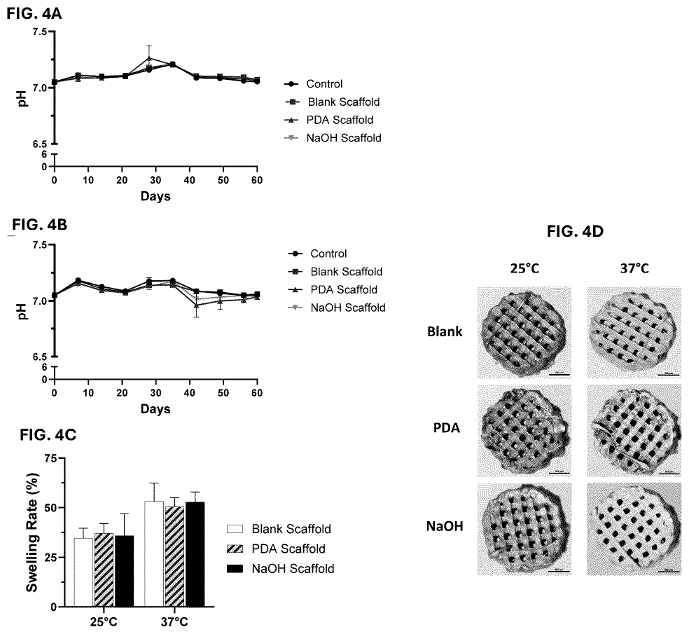

Degradation of 3D-printed PLA scaffolds was monitored for 60 days while monitoring the pH of the surrounding media (PBS or water), changes in weight, swelling rate, and contact angle (FIG. 4 and Table 2). At 4° C., no observable degradation occurred when the scaffolds were incubated in either PBS or water (data not shown). Degradation studies at 25° C. and 37° C. did not lead to significant changes in pH (FIG. 4A,B). The scaffold swelling rate was dependent on the temperature of incubation and not surface treatment (FIG. 4C). The contact angle measured on day 60 of in vitro degradation (with incubation at 37° C.) showed a marked decrease for Blank scaffolds from 87.5±7.8 to 74.7±9.9 (on day 60) (Tables 1 and 2). For NaOH scaffolds, the contact angle decreased from 74.5±9.9 to 62.7±5.1 on day 60 of the degradation studies. We did not observe a similar trend of decrease in contact angle with PDA scaffolds following 60 days of degradation studies (Tables 1 and 2). After examining the images of the scaffolds after 60 days, we did not observe significant differences in the scaffolds incubated at 25° C.; however, a light color was observed in scaffolds incubated at 37° C. in comparison to 25° C. (FIG. 4D).

### Degradation Biocompatibility

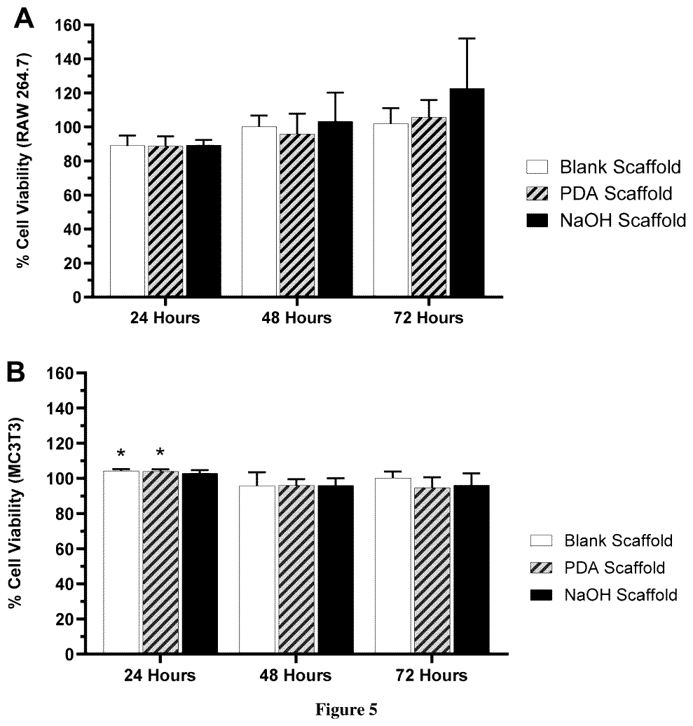

The biocompatibility of degraded scaffolds was assessed using aliquots of water collected on day 60 of the degradation studies. The aliquots were mixed with cell culture media at the ratio of 1:5 (aliquot: cell media) and incubated (37° C.) for 24, 48, and 72 h (FIG. 5). Cell viability was compared to untreated control cells with regular growth media. Overall, the degraded scaffolds did not affect the viability of pre-osteoclast or pre-osteoblast cells (FIG. 5).

### Osteoclast Differentiation and Functionality

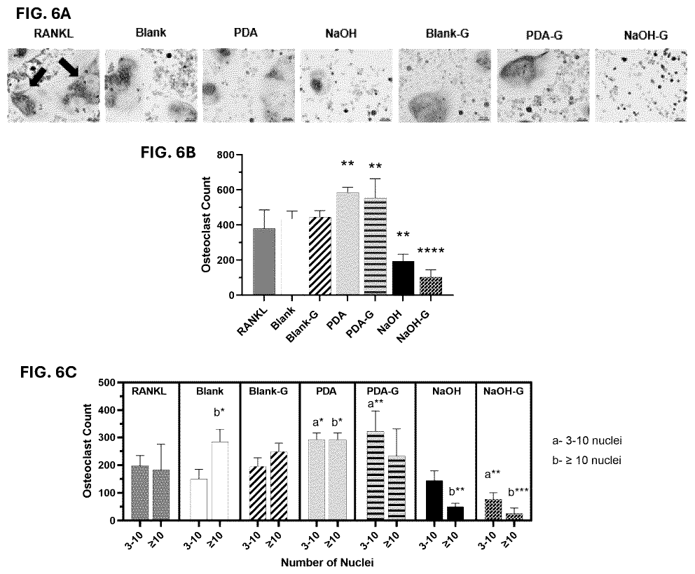

The overall efficacy of the GaA cAc-loaded scaffolds was studied using differentiating pre-osteoclastic cells (RAW 264.7), with TRAP-stained osteoclasts (pink cells with ≥3 nuclei) being imaged and differentially counted based on the number of nuclei (FIG. 6). We observed that Blank scaffolds, as well as PDA scaffolds without GaAcAc loading, supported osteoclast differentiation as shown in the representative images (FIG. 6A). For NaOH scaffolds, we observed a reduced number of multi-nucleated cells (osteoclasts) (FIG. 6A). The osteoclast count did not show detectable changes for Blank scaffolds with or without GaAcAc loading when compared to positive control cells that received only RANKL (FIG. 6B). PDA and PDA-G scaffolds showed an increase in osteoclast count versus positive control cells that received only RANKL treatment (p<0.01, 0.0001; FIG. 6B). We noticed a pronounced reduction in the number of osteoclasts for NaOH-scaffolds irrespective of GaAcAc loading status (p<0.0001; FIG. 6B). The trend prompted us to conduct a differential count of osteoclasts according to number of nuclei (FIG. 6C). For Blank scaffolds, compared to positive control, we observed an increase in the number of osteoclasts with ≥10 nuclei (p<0.01; FIG. 6C). However, GaAcAc loading onto Blank scaffolds did not lead to detectable changes in osteoclast differential count (FIG. 6C). When compared to positive control cells (RANKL treatment only), PDA scaffolds resulted in an increase in osteoclasts at the 3-10 and ≥10 nuclei categories. In contrast, PDA-G scaffolds caused a slight reduction (not significant) in osteoclast count at ≥10 nuclei category. There was a considerable reduction in osteoclasts with ≥10 nuclei with NaOH and NaOH-G scaffolds (p<0.01, 0.001; FIG. 6C).

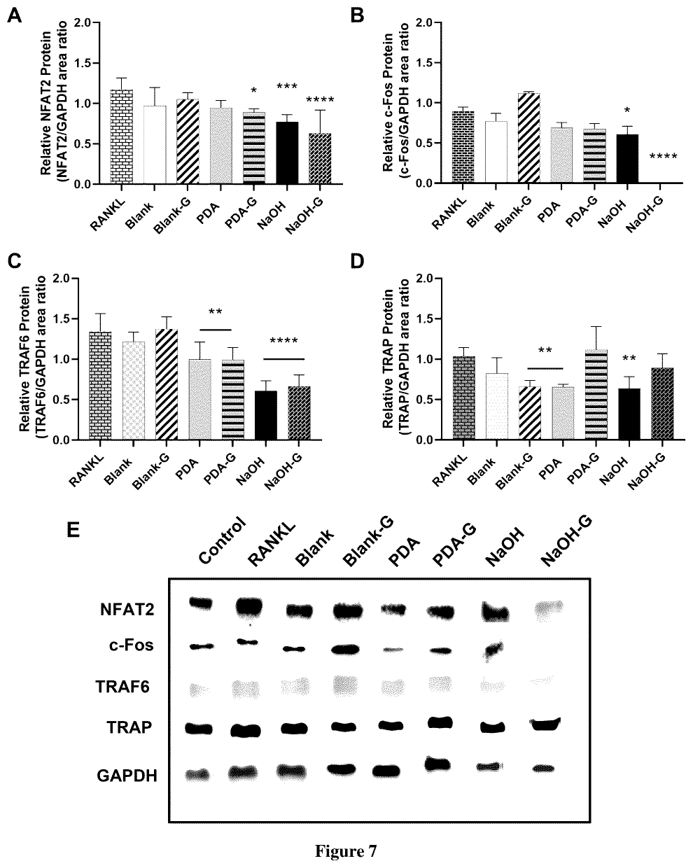

To confirm this trend, a western blot analysis was conducted focusing on the expression of key osteoclast differentiation markers NFAT2, c-Fos, TRAF6, and TRAP (FIG. 7). In alignment with the previous experiment, treatment with NaOH and NaOH-G scaffolds resulted in a marked decrease in the expression of all factors, including a complete knockout of c-Fos expression in cells cultured with NaOH-G scaffolds (p<0.0001, FIG. 7B). Cells treated with PDA-G scaffolds showed decreased expression of NFAT2 and TRAF6, but c-Fos and TRAP expression remained unaffected (p<0.05, 0.01; FIG. 7A-D).

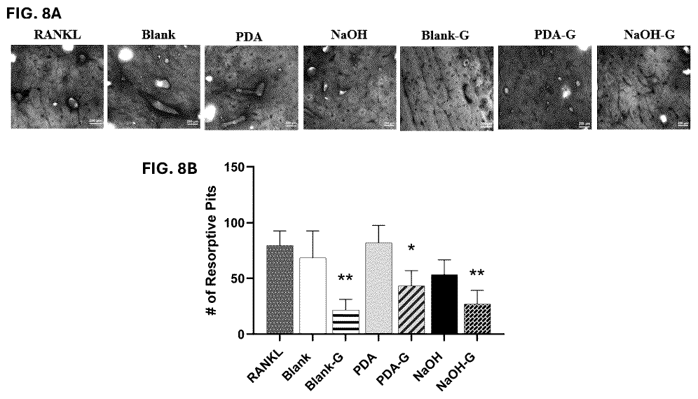

Further, osteoclast differentiation studies were performed on cortical bovine bone slices to assess functionality. After osteoclast differentiation, the bone slices were initially TRAP-stained and stained with toluidine blue to visualize the resorptive pits. The number of resorptive pits with Blank scaffolds was comparable to positive control cells (RANKL treatment only) (FIG. 8). Similarly, PDA and NaOH scaffolds did not cause significant reduction in the number of resorptive pits (FIG. 8B). All scaffolds loaded with GaAcAc (Blank-G, PDA-G and NaOH-G) exhibited significantly fewer resorptive pits compared to scaffolds without GaAcAc (p<0.05, 0.01; FIG. 8B).

### Discussion

Therapeutic management of bone disorders will benefit tremendously from suitable delivery systems that will facilitate retention of osteogenic therapeutics at the site of action, prevent off-target distribution, and maximize therapeutic effects at minimum effective doses. Three-dimensional printing technology is gaining so much attention because of the opportunity to customize treatment for individual patients. We recently reported the efficacy of a new gallium compound, GaAcAc in inhibiting osteoclastic bone resorption (Ghanta P. et al., Drug Deliv Transl Res. 2023 October; 13(10):2533-2549). This work has examined the potential utility of 3D-printed scaffolds to deliver GaAcAc. We adopted a fused deposition-based 3D printing technology that applies a thermoplastic polymer filament such as PLA, which is known to be biodegradable and biocompatible and has been applied in various biomedical applications. We selected the 2 mm diameter over 4 mm scaffolds since they showed the best degradation profiles.

We avoided loading GaAcAc onto the polymer filament before scaffold printing as it would have subjected the drug to the elevated temperature at which the scaffolds were printed. Thus, we considered post-manufacture strategies to load GaAcAc onto printed scaffolds (FIG. 1). For Blank scaffolds that did not receive any surface pretreatment, we anticipated that the acid end groups of PLA polymers would confer a negative charge that could be favorable for GaAcAc loading. It is also likely that the porous nature of the scaffolds could favor GaA cAc loading onto Blank scaffolds. Further, we modified the surface of scaffolds with PDA or NaOH and conducted GaA cAc loading. The overall trend indicated that we were able to load GaAcAc onto the different types of scaffolds with NaOH surface treatment showing the highest GaAcAc loading (p<0.05; FIG. 1). Additionally, for all scaffolds loaded with GaAcAc, we observed characteristic bands around 3384 wavenumber cm⁻¹ that could be attributed to the acetylacetonate ligand associated with Ga3b. FTIR spectra also suggested that the surface pretreatment with PDA or NaOH was very light and did not cause significant changes in the transmittance of IR signal (FIG. 2).

In addition to increased GaAcAc loading capabilities, the surface treatments also favorably affected the hydrophilic properties of the scaffolds (Table 1). Contact angle is a commonly used measurement for hydrophilicity, with higher angles being associated with hydrophobic properties, indicating potential negative effects on cell adhesion and proliferation (Menzies KL and Jones L, Optom Vis Sci. 2010 June; 87(6):387-99). In alignment with previous studies, both surface modifications increased the hydrophilicity of the scaffolds, with PDA exhibiting the lowest contact angle (Jaidev LR and Chatterjee K, Mater Des. 2019 January 161(5):44-54). Furthermore, the biocompatibility studies showed comparable trends among all the scaffolds with or without surface pretreatments (FIG. 3). The in vitro biodegradation studies were conducted for up to 60 days while monitoring potential changes in the pH that could arise from possible production of lactic acid from PLA (Xiao RZ et al. Int J Nanomedicine. 2010 Nov. 26; 5:1057-65). There was no detectable impact of scaffold surface treatment on swelling rate studies conducted following day 60 of the degradation studies.

We evaluated the efficacy of the 3D-printed scaffolds in osteoclast differentiation using RAW 264.7 cells as pre-osteoclast cells (Ghanta P. et al., Drug Deliv Transl Res. 2023 October; 13(10):2533-2549). Thus, the differentiation process required the addition of receptor activation of nuclear factor kappa-B ligand (RANKL) to achieve mature, multinucleated osteoclasts (Ghanta P. et al., Drug Deliv Transl Res. 2023 October; 13(10):2533-2549). Compared to positive control cells that received RANKL alone, cells incubated with Blank scaffolds differentiated similarly, as shown in the comparable number of mature osteoclasts (FIG. 6). We observed differences in osteoclast differentiation among scaffolds based on surface pretreatments, with PDA scaffolds showing significantly higher osteoclast counts (p<0.001) than both Blank and NaOH scaffolds, which could be due to the enhanced hydrophilicity, cell attachment with the PDA-treated scaffolds as observed in FIG. 3 which is in agreement with earlier report (Chakka JL et al., RSC Adv. 2021 Apr. 11 (22):13282-13291). There was no detectable difference in osteoclast counts between Blank scaffolds with or without GaAcAc loading (FIG. 6B). A similar trend was observed with PDA scaffolds (PDA versus PDA-G) (FIG. 6B). Meanwhile, GaAcAc loading onto scaffolds that received surface pretreatment with NaOH showed further reduction in the number of osteoclasts (NaOH versus NaOH-G, FIG. 7B). The effects of NaOH-G scaffolds on osteoclast count could likely be linked to our observation that NaOH treatment resulted in the highest amount of GaAcAc loading compared to other types of scaffolds (FIG. 1C; p<0.05). It is also possible that NaOH used in scaffold pretreatment affected osteoclast differentiation and function by causing a small shift in extracellular pH that have been shown in earlier studies to affect osteoclasts (Arnett T and Spowage M, Bone. 1996 March; 18(3):277-9). It was reported that small and large resorbing osteoclasts had significantly higher basal pH than their non resorbing counterparts (Lees RL et al. Bone. 2001 February; 28(2):187-94). Focusing on scaffold loaded with GaAcAc, our findings remain in alignment with previous studies investigating the osteoclast inhibitory effects of gallium compound-loaded biomaterials, (Qiu C. et al., Ceram Int. 2020 July 46(10):16364-16371), however, to our knowledge, this is the first study to investigate GaA cAc loading in 3D printed PLA scaffolds and the investigation of the impact of scaffold surface pretreatments.

To analyze the mechanistic basis of the observed trends in osteoclast differentiation, we performed western blotting analyses focusing on critical markers involved in the initial process of osteoclast differentiation, such as NFAT2, c-Fos, TRAF6, and TRAP (FIG. 7). Surface pretreatment of scaffolds and GaAcAc loading did lead to a significant reduction in osteoclast differentiation markers. For instance, Blank scaffolds showed no detectable difference in levels of all markers compared to positive control cells that received RANKL alone. Meanwhile, PDA scaffolds showed a reduction in TRAF6 and NaOH scaffolds led to a reduction in all the differentiation markers (p<0.01). Among scaffolds loaded with GaAcAc, NaOH-G resulted in a reduction of all the differentiation markers while PDA-G led to significant downregulation of NFAT2 and TRAF6 OC markers (p<0.0001; FIG. 7). The impact of scaffold surface pretreatment and GaAcAc was pronounced from osteoclast function studies based on the extent of resorption of bovine bone slices. In the experiment, we conducted osteoclast differentiation incubated with different scaffolds. The time at which osteoclast function is impeded will translate into a reduction in the number of resorption pits. We observed that Blank scaffolds did not interfere with osteoclast function. Similarly, scaffolds that received PDA and NaOH surface pretreatments did not interfere with bone resorption. Each scaffold without GaAcAc loading showed a comparable number of resorptive pits to the positive control. All the scaffolds with GaA cAc loading significantly reduced the number of resorptive pits compared to positive controls that received RANKL alone (p<0.01). Compared to the RANKL group (positive control), Blank-G reduced the number of resorptive pits by 3.7 times, followed by NaOH-G (2.9 times reduction in resorptive pits) while PDA-G caused a 1.8 times reduction in the number of resorptive pits.

In conclusion, our work demonstrated assessment of different methods to prepare GaAcAc-loaded 3D-printed PLA scaffolds for potential application in osteoclastic bone resorptive disorders. Highlights on 3D-printed scaffold surface pretreatment with PDA or NaOH include: (a) no observable effects on biocompatibility or biodegradation parameters, (b) a modest effect on GaAcAc loading with NaOH scaffolds having the highest concentration of GaAcAc, and (c) a marked increase in scaffold hydrophilicity. Our data showed that surface-modified 3D-printed PLA scaffold-loaded with GaAcAc significantly decreased OC differentiation most likely at an early stage of OC differentiation, which was further confirmed by the mechanistic studies where there was a significant downregulation in NFAT, c-Fos & TRAF6 expression. Overall, we observed that GaAcAc-loaded scaffolds with NaOH pretreatment showed pronounced effects in suppressing osteoclast differentiation markers and osteoclast count. It is likely that NaOH pretreatment had synergistic effects with GaAcAc through modulation of extracellular pH that possibly affected osteoclast differentiation (Lees RL et al. Bone. 2001 February; 28(2):187-94). In osteoclast function assessment via bone resorption, it is noteworthy that only GaAcAc-loaded scaffolds inhibited the bone resorption as measured by the number of resorptive pits. The ability of GaAcAc-loaded scaffolds to reduce the number of osteoclastic resorptive pits was found to follow this ranking: Blank G>NaOH-G>PDA-G. Additional studies will be needed to fully elucidate the impact of scaffold surface treatment on GaAcAc loading and efficacy in osteoclastic bone resorption.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A biocompatible 3D-printed scaffold, comprising a biocompatible polymer shaped to form a scaffold using 3D printing and a gallium compound.

2. The biocompatible 3D-printed scaffold of claim 1, wherein the gallium compound is selected from the group consisting of gallium acetylacetonate, gallium nitrate, gallium citrate, gallium maltolate, gallium carbonate, gallium acetate, gallium triacetate, gallium tartrate, gallium oxide, gallium hydroxide, and hydrated gallium oxide.

3. The biocompatible 3D-printed scaffold of claim 1, wherein the gallium compound is gallium acetylacetonate.

4. The biocompatible 3D-printed scaffold of claim 1, wherein the biocompatible polymer is polylactide.

5. The biocompatible 3D-printed scaffold of claim 1, wherein the scaffold is a bone scaffold.

6. The biocompatible 3D-printed scaffold of claim 1, wherein the gallium compound has been loaded onto the surface of the 3D-printed polylactic acid scaffold.

7. The biocompatible 3D-printed scaffold of claim 1, wherein the gallium compound is evenly loaded onto the 3D-printed scaffold.

8. A method of inhibiting bone resorption in a subject, comprising implanting a biocompatible 3D-printed scaffold comprising a biocompatible polymer shaped to form a scaffold using 3D printing and a therapeutically effective amount of a gallium compound into the subject.

9. The method of inhibiting bone resorption of claim 8, wherein the subject has been diagnosed as having a bone growth disease or disorder.

10. The method of claim 9, wherein the subject has been diagnosed as having bone growth disease or disorder selected from the group consisting of osteogenesis imperfecta, disorders caused by increased osteoclastogenesis or bone loss associated with inflammatory conditions, infection, genetic and age-related bone disorders such as osteoporosis, osteopenia, Paget's disease, metastatic bone cancer, myeloma bone disease, bone fracture healing, and bone graft repair.

11. The method of claim 8, wherein the scaffold is a bone scaffold.

12. The method of claim 8, wherein the gallium compound is gallium acetylacetonate.

13. The method of claim 8, wherein the biocompatible polymer is polylactic acid.

14. The method of claim 8, wherein the biocompatible 3D-printed scaffold stimulates osteoclast differentiation.

15. The method of claim 8, wherein the gallium compound is released from the 3D-printed scaffold in a sustained release manner.

16. A method of making a biocompatible 3D-printed scaffold, comprising preparing a 3D-printed scaffold comprising a biocompatible polymer using a 3D printing method; and loading the surface of the 3D-printed scaffold with a gallium compound.

17. The method of claim 16, wherein the 3D printing method is fused deposition modeling.

18. The method of claim 16, wherein the surface of the 3D-printed scaffold is treated with polydopamine or sodium hydroxide before loading the surface of the 3D-printed scaffold with the gallium compound.

19. The method of claim 16, wherein the biocompatible polymer is polylactic acid.

20. The method of claim 16, wherein the gallium compound is gallium acetylacetonate.

21. The method of claim 16, wherein the gallium compound is evenly loaded onto the 3D-printed scaffold.
